(19) United States
(12) Patent Application Publication
(10) Pub. No.: US 2013/0243810 A1
(43) Pub. Date: Sep. 19, 2013

(54) SYNERGISTIC IMMUNOGENIC COMPOSITIONS BASED ON PROTEIN ANTIGENS COMBINED WITH PERTUSSIS CELL ANTIGEN AND INACTIVATED TOXINS

(71) Applicants: Eliane Namie Miyaji, Sao Paulo (BR); Isaias Raw, Sao Paulo (BR); Maria Leonor Sarno de Oliveira, Sao Paulo (BR); Paulo Lee Ho, Sao Paulo (BR)

(72) Inventors: Eliane Namie Miyaji, Sao Paulo (BR); Isaias Raw, Sao Paulo (BR); Maria Leonor Sarno de Oliveira, Sao Paulo (BR); Paulo Lee Ho, Sao Paulo (BR)

(21) Appl. No.: 13/876,746

(22) PCT Filed: Sep. 26, 2011
(86) PCT No.: PCT/BR11/00350

§ 371 Date: Jun. 7, 2013

(30) Foreign Application Priority Data
Sep. 28, 2010 (BR) PI 1003753-5

(51) Int. Cl. A61K 39/116

(52) U.S. Cl. CPC A61K 39/116
USPC 424/192.1; 424/203.1

(57) ABSTRACT

The present invention relates to synergistic immunogenic compositions for preventing whooping cough and infections caused by Streptococcus pneumoniae. Additionally, the compositions according to the present invention may protect against infections caused by other pathogenic agents by means of a synergistic combination of antigens thereof. In particular, the present invention provides synergistic immunogenic compositions that comprise at least one cell antigen of inactivated Bordetella pertussis and at least one protein antigen comprising one or more surface proteins A (PspA) of Streptococcus penumoniae or fragments thereof. The present invention further provides synergistic immunogenic compositions comprising at least one cell antigen of inactivated Bordetella pertussis, at least one protein antigen comprising one or more surface proteins A (PspA) of Streptococcus penumoniae or fragments thereof, and one or more diphtheria and/or tetanus toxins. The present invention also relates to the use of one or more synergistic immunogenic compositions according to the present invention for producing combined vaccines for the prevention of whooping cough, tetanus, diphtheria and/or infections caused by Streptococcus pneumoniae. The present invention further relates to the use of the compositions according to the present invention for producing vaccines for the prevention of whooping cough, tetanus, diphtheria and/or infections caused by Streptococcus pneumoniae.

### FIELD OF THE INVENTION

The present invention relates to synergistic immunogenic compositions for preventing whooping cough and infections caused by Streptococcus pneumoniae. Additionally, the compositions according to the present invention may protect against infections caused by other pathogenic agents by means of a synergistic combination of antigens thereof.

In particular, the present invention provides synergistic immunogenic compositions that comprise at least one cell antigen of inactivated Bordetella pertussis and at least one protein antigen comprising one or more surface protein A (PspA) of Streptococcus penumoniae or fragments thereof.

The present invention further provides synergistic immunogenic compositions comprising at least one cell antigen of inactivated Bordetella pertussis, at least one protein antigen comprising one or more surface protein A (PspA) of Streptococcus penumoniae or fragments thereof, and one or more diphtheria and/or tetanus toxins,

The present invention also relates to the use of one or more synergistic immunogenic compositions according to the present invention for producing- combined vaccines for the prevention of whooping cough, tetanus, diphtheria and/or infections caused by Streptococcus pneumoniae.

The present invention further relates to the use of the compositions according to the present invention for producing vaccines for the prevention of whooping cough, tetanus, diphtheria and/or infections caused by Streptococcus pneumoniae.

### BACKGROUND OF THE INVENTION

Streptococcus pneumoniae Vaccines

Streptococcus pneumoniae (pneumococcus) is one of the major causative agents of respiratory diseases which, depending on some factors such as age and immune status of the subject, may evolve into a systemic condition or severe disease such as meningitis. Pneumococcal diseases kill approximately 800,000 children per year worldwide. In developing countries, deaths caused by these infections account for 5% of total childhood deaths annually. Moreover, the pneumococcus causes upper respiratory tract diseases with, high incidence in. children which are less severe, such as otitis media and sinusitis.

The polysaccharide capsule surrounding the pneumococcus is highly variable and determines the bacterium classification in more than 90 serotypes. While there is no cross-reactivity among each other, the capsular polysaccharides are currently the only commercially available active ingredients of vaccines against S. pneumonise.

Pneurnovax 23 (Merck & Co., Inc.) is a vaccine comprising capsular polysaccharides of 23 pneumococcal serotypes prevalent in the northern hemisphere. In addition to the complexity of its production, which involves the fermentation of 23 pneumococcal strains and purification of the polysaccharides thereof, this vaccine is not very effective in children under two years old and in elderly people, two groups at risk for pneumococcal diseases.

In the case of Prevnar (PCV7, Wyeth Pharmaceuticals), it is a vaccine comprising polysaccharides of 7 pneumococcal serotypes prevalent in. the United States which are conjugated to a protein component, diphtheria toxoid CRM₁₉₇. By action of said protein component, this vaccine is effective in children under two years old and in elderly people, but it has limited coverage varying in accordance with the serotypes circulating in different parts of the world. In Brazil, the estimated coverage afforded by this vaccine is about 52% (Brandileone MCC. et al. The Journal of Infectious Diseases 2003, 187:1206-1212).

New versions of conjugate vaccines comprise polysaccharides of 10 or 13 different serotypes and significantly increase the coverage against serotypes that cause diseases. However, some studies carried out after the introduction of the PCV7 vaccine in different countries show an increase of diseases caused by serotypes not present in the vaccines (Ghaffar F. et al. Clinical Infectious Diseases 2004; 39:930-938; Jacobs M R. et al. Laryngoscope 2007; 117:295-298; Kaplan S L. et al, Pediatrics 2010; 125:429-436), which may also occur with vaccines comprising 10 or 13 polysaccharides. Thus, the high manufacturing costs and the potential serotype replacement a few years after the introduction of vaccination are factors that lead different research groups to study new alternatives in this field.

Over the last decade, much has been learned about the virulence factors of pneumococci and the role of each of them during the course of disease (Tai S S. Critical Reviews in Microbiology 2006, 32:139-153). Besides the polysaccharide capsule that covers the bacterium surface and takes part in the immune system evasion, several proteins protrude to the outside and thus interact with epithelial and endothelial cells, promoting adhesion and pathogen invasion, and with immune system components.

Within this definition are the different variants of the “Pneumococcal Surface Protein A” (PspA), which are among the most promising vaccine candidates proposed. Their protective potential has been demonstrated in different presentation models and animal challenges (Briles B E. et al. Vaccine 2000; 18:1707-1711; Briles D E, et al. Infection and Immunity 2000; 68:796-800; Arêas APM, et al. Infection and Immunity 2005; 733810-3813; Ogunniyi A D. et al. Infection and Immunity 2007; 75:350-357; Ferreira D M. et al. Clinical and Vaccine Immunology 2008; 15:499-505; patent application no. PI 9908649-2 A2, patent application no. PI 0011478-2 A2, U.S. Pat. No. 5,679,768 and U.S. Pat. No. 5,804,193).

PspA proteins have a molecular weight between 67 and 99 kDa (Waltman W D. et al , Microbial Pathogenesis 1990; 8:61-69) and they are divided into four regions: the highly loaded α-helix N-terminal region, the region rich in proline residues, the choline binding region and a C-terminal tail of 17 amino acids with hydrophobic character. Based on the amino acid sequence of the region immediately preceding the region rich in proline residues (CDR—“Clade-Defining Region”), the PspA were grouped into 6 clades which are separated into three families (Hollingshead S. et al. Infection and Immunity 2 000; 68:5889-5900).

One of the problems related to the selection of PspA as a vaccine component is the relative variability that such protein presents in several isolates (Hollingshead, S. et al. Infection and Immunity 2000; 68:5889-5900). Isolate data obtained from different parts of the world show that more than 90% of isolates express PspA belonging to families 1 and 2 (Briles B E. et al. Vaccine 2000; 18:1707-1711). In South America, studies carried out in Brazil (Brandileone MCC. et al. Vaccine 2004; 22:3890-3896) and Colombia (Vela Coral MC. et al. Emerging Infectious Diseases 2001; 7:832-836) show similar results, with the prevalence of strains with PspA belonging to families 1 and 2 in 94% and 97.5% of isolates, respectively. It was proposed that an ideal vaccine comprising PspA should contain a representative of family 1 and a representative of family 2 due to the cross-reactivity between members of the same family (patent application no. PI 9908649-2 A2). Thus, such, vaccine would theoretically cover approximately 90% of the isolates. However, this theory does not hold up, as new data point out the molecules which have low cross-reactivity with others of the same family. This factor has led to the cross-reactivity study of antibodies produced against the N-terminal region of PspA from different clades using several pneumococcal serotypes isolated in Brazil. It was demonstrated that PspA molecules of clades 4 and 5 (PspA4 and PspA5) are able to induce antibodies that, recognize different PspAs, even if they belong to different families (Darrieux M. et al. Journal of Medical Microbiology 2008; 57:273-278). In the same study, a cross-reactivity analysis of animal sera which were previously immunized with hybrid PspA comprising fused PspA fragments from different families, particularly PspA N-terminal regions of clades 1 and 4, has demonstrated a strong recognition, of isolates comprising clades belonging to families 1 and 2. Furthermore, mice immunized with PspA4 or PspA5 showed, protection, against lethal intranasal, challenge with pneumococcal isolates expressing PspA of these two families (Moreno A T. et al. Immunology Clinical and Vaccine 2010; 17:439-446). Thus, PspA4 and PspA5 proteins combined or not with PspA1 or fragments thereof, appear to be the selected antigens for a vaccine which presents broad isolate coverage.

Cell Pertussis Vaccine

Bordetella pertussisis a bacterium which causes an infectious disease of the respiratory tract known as whooping cough or pertussis. The cell pertussis vaccine (PV) produced by Instituto Butantan comprises B. pertussis bacteria inactivated by treatment with formalin. This vaccine is given to children in combination with diphtheria and tetanus toxoids in a formulation known as DTP, which protects against diphtheria, tetanus and pertussis. After its introduction in the country, there was a rapid and significant reduction, in the incidence of pertussis cases (de Carvaiho AP. & Pereira EM. Jornal de Pediatria 2006; 82:S15-S24), Previously, cell pertussis vaccine was successfully adopted by several countries, however, some adverse effects probably related, to impurities derived from inactivation methods, which vary in different countries, or the amount of lipopolysaccharide endotoxin (LPS), led to the replacement thereof by acellular formulations (Locht C. Microbes and Infection 2008; 10:1051-1056). In Brazil, there are no reports of severe adverse effects related to cell pertussis vaccine produced by Instituto Butantan, which has been administered to two-month-year-old children and older for more than 20 years. Cell, vaccines are also adopted by other developing countries since acellular vaccines are generally less effective and, above all, present high manufacturing costs (Locht C. Microbes and Infection 2008; 10:1051-1056).

Recently, Institute Butantan developed a technology for removing most of the LPS of PV and producing the new cell pertussis vaccine “low” (PV_{L}) (patent application no. PI0402630-6 A). Such new vaccine has been successfully tested, in phase 1 clinical trials and it showed, results which are similar to conventional cell vaccine PV (Zorzeto T Q. et al. Clinical and Vaccine Immunology 2009; 16:544-550).

Combined Vaccines

Combined vaccines offer the advantage of a small number of inoculations in the patient, also resulting in lower costs associated with the administration of immunobiologicals. The combination of a vaccine comprised by an inactivated whole bacterium with a protein antigen from another pathogenic organism can maximize the immune response against this second component via an adjuvant or synergistic effect.

Combination of Cell Pertussis Vaccine and PspA

Although the literature data point out to the PspA antigen as an antigen with high potential in the development of vaccines against infections caused by pneumococcal, all approaches to date are far from being ideal. Factors such as number of doses, immunization route and strategic suitability for use in newborn or few-month-year-old children should be considered for a vaccine proposal which is effective and presents low cost, safety and embraces the whole population.

The combination of antigens with different adjuvants quantitatively and qualitatively modulates the immune response, which decisively influences the above mentioned factors and the success of a vaccine (Reed S G. et al. Trends in Immunology 2008; 30:23-32). In general, vaccines comprised by inactivated or attenuated pathogens such as cell pertussis vaccine, have inherent immunomodulatory properties which avoids the use of other adjuvants. Bacterial components such as lipopolysaccharides, lipoteichoic acids, proteins and nucleic acids are known recognition receptor ligands of molecular patterns associated with pathogenic organisms. These receptors are present on the surfaces and also inside the host cells and the activation thereof has stimulatory effects on the immune system (Reed S G. et al. Trends in Immunology 2008; 30:23-32; Harandi A M. et al. Expert Reviews of Vaccines 2009; 8:293-298).

There are prior art examples of vaccines which use the immunomodulatory property of cellular adjuvants in combination with other antigens. An example is the combination of a cell pertussis vaccine with an influenza vaccine by nasal route, which was proposed, by Berstad A K. et al. Journal of Medical Microbiology 2000; 49:157-163. Another example is a vaccine against pleural pneumonia in swine comprising cell components or antigens isolated from Haemophilus pleuropneumoniae combined with B. pertussis adjuvant, which is described in the International Publication no. WO 80/02113 of Oct. 16, 1980.

### OBJECTS OF THE INVENTION

It is an object of the present invention to provide synergistic immunogenic compositions comprising at least one cell antigen of inactivated Bordetella pertussis and at least one protein antigen comprising one or more surface protein A (PspA) of Streptococcus penumoniae or fragments thereof and a pharmaceutically acceptable carrier. It is another object of the present invention to provide synergistic immunogenic compositions comprising at least one cell antigen of inactivated Bordetella pertussis, at least one protein antigen comprising one or more surface protein A (PspA) of Streptococcus penumoniae or fragments thereof, one or more inactivated diphtheria toxins and a pharmaceutically acceptable carrier.

It is another object of the present invention to provide synergistic immunogenic compositions comprising at least one cell antigen of inactivated Bordetella pertussis, at least one protein antigen comprising one or more surface protein A (PspA) of Streptococcus penumoniae or fragments thereof, one or more inactivated tetanus toxins and a pharmaceutically acceptable carrier.

It is a further object of the present invention the use of one or more synergistic immunogenic compositions for producing combined vaccines for the prevention of whooping cough, tetanus, diphtheria, and/or infections caused by Streptococcus pneumoniae.

### DESCRIPTION OF THE DRAWINGS

The following drawings are part of the present specification and. are included herewith in order to illustrate certain aspects of the invention. The object of the present invention may be better understood by reference to one or more of these drawings in combination with the detailed description of the preferred embodiment presented herein.

FIG. 1 shows the amount of pathogen (pneumococcal) recovered from lung lavage (A) and blood (B) from. BALB/c mice immunized with PV or PspA5-PV collected at different time after intranasal challenge with the ATCC6303 isolate. Colony-forming units (CPU) were quantified, after plating samples of 4 animals per group on blood agar. The triangles represent mice individual samples and the lines indicate the average of each group (d=days).

FIG. 2 shows the induction of anti-PspA5 antibodies in mice intranasally immunized with different vaccine formulations. Three weeks after the last immunization, anti-PspA IgG antibodies were detected in serum (A) by ELISA. IgG (B) and IgA (C) anti-PspA antibodies were also detected in bronchoalveolar fluid (BALF) samples. The average antibody concentration of 6 (A) and 4 (B and C) animals is indicated. Asterisks indicate statistically significant differences in the control group or in the group immunized with PspA5 (*P<0.05, **P<0.005, Mann-Whitney U test).

FIG. 3 shows the cross-reactivity induced by immunization with PspA5-PV. A total protein lysate from different pneumococcal isolates was analyzed by “immunoblotting” using a 1:500 dilution of serum from BALB/c mice intranasally immunized with PspA5 (A) or PspA5-PV (B).

FIG. 4 shows the induction of anti-PspA5 antibodies in mice subcutaneouslly immunized with different vaccine formulations. Three weeks after immunization, anti-PspA5 IgG antibodies were detected in serum by ELISA. The average antibody concentration of 6 animals per group is shown. The results are representative of two independent experiments. Asterisks indicate significant differences among the indicated groups (**P<0.005, comparison among non-immunized mice or with respective control, and *P<0.01, comparison among mice immunized with PspA5, Mann-Whitney U test).

### DETAILED DESCRIPTION OF THE INVENTION

Description of the Synergistic immunogenic Compositions

The present invention relates to synergistic immunogenic compositions comprising at least one cell antigen of inactivated Bordetella pertussis and at least one protein antigen comprising one or more surface protein A (PspA) of Streptococcus penumoniae for protection against pertussis and infections caused by Streptococcus pneumoniae.

The synergistic immunogenic compositions of the present invention comprise at least one cell antigen of inactivated Bordetella percussis and at least one protein antigen comprising one or more surface protein A (PspA) of Streptococcus pneumoniae and a pharmaceutically acceptable carrier.

Preferably, the cell antigen of inactivated Bordetella pertussis comprises low content of lipopolysaccharides. This is due to the fact that the lipopolysaccharide of Gram negative bacteria wall to be an endotoxin with toxic, inflammatory and pyrogenic activity. The partial removal thereof from Bordetella pertussis can be performed by extraction with suitable solvents and detergents, resulting in a cell antigen of Bordetella pertussis with low content of lipopolysaccharides (VP_{L}).

Preferably, the protein antigen is a combination of at least two PspA, combined fragments thereof or fused fragments thereof selected from clades belonging to families 1 and 2. Preferably, the PspA or fragments thereof are selected from clades 1, 4 and 5.

Preferably, the PspA fragments are selected from the α-helix N-terminal region to the region rich in proline residues. More preferably, the PspA fragment is selected from the region rich in proline residues.

The synergistic immunogenic compositions of the present invention may further comprise one or more antigens of inactivated toxins, Preferably, the inactivated toxins are selected from inactivated diphtheria toxin and inactivated tetanus toxin.

Thus, the synergistic immunogenic compositions of the present invention may comprise at least one cell antigen, of inactivated Bordetella pertussis, at least one protein antigen comprising one or more surface protein A (PspA) of Streptococcus pneumoniae or fragments thereof, one or more inactivated diphtheria and/or tetanus toxin and a pharmaceutically acceptable carrier.

The inactivated components of the synergistic immunogenic compositions of the present invention may be obtained by any method known in the art, such as chemical procedures, treatment with formaldehyde or hydrogen peroxide or even recombinant DNA techniques.

According to the present invention, “adjuvants” are molecules, components, macromolecules or attenuated or killed microorganisms which enhance the immune response, reduce the amount of requested antigen and direct the type of immune response to be developed, besides holding it for a higher period of time as an immunogen, being any material or substance that changes the type, speed, intensity or duration of the immune response.

The compositions of the present invention can further comprise excipients, such as bactericides, bacteriostats, antioxidants, preservatives, buffers, stabilizers, pH adjusters, osmolarity adjusters, antifoaming agents and surfactants; and residual inactivating or fractioning agents, growth medium components and solvents commonly used in vaccine production. Examples of these types of components can be found in Epidemiology and Prevention of Vaccine-Preventable Diseases The Pink Book, 11th edition, “Vaccine Excipient & Media Summary” section (Centers for Disease Control and Prevention, Epidemiology & Prevention of vaccine-preventable Diseases. Atkinson W, Wolfe S, Hamborsky J, McIntyre L, eds. 11th ed, Washington D.C.: Public Health Foundation, 2009) incorporated herein by reference.

As used in the present invention, the use of the expression “pharmaceutically acceptable” means a non-toxic solid, inert, semi-solid liquid excipient, diluent, auxiliary formulation of any type or simply a sterile aqueous medium, such as saline. Some examples of materials which can act as pharmaceutically acceptable carriers are sugars such as lactose, glucose and sucrose, starches such as corn starch and potato starch, cellulose and derivatives thereof such as sodium carboxymethylcellulose, ethyl cellulose and cellulose acetate, cyclodextrin; oils such as peanut oil, cottonseed oil, sunflower oil, sesame oil, olive oil, corn oil and soybean oil; glycols such as propylene glycol, polyols such as glycerin glycol, sorbitol, mannitol. and polyethylene; esters such as ethyl laurate, ethyl oleate, agar; buffering agents such as aluminum hydroxide and. magnesium hydroxide; alginic acid, pyrogen free water; isotonic saline, Ringer's solution; buffer solutions of ethyl alcohol and phosphate, as well as other compatible non-toxic substances used in pharmaceutical formulations.

A number of administration routes of the immunotherapeutic compositions and vaccines described in the present invention is available. The particular selected mode will depend on the particular selected active ingredient, the required dosage for therapeutic efficacy and the patient to whom the composition is administered. The methods of the present invention can generally be practiced using any biologically acceptable administration route, i.e. any route that produces effective levels of an immune response without causing undesirable clinically adverse reactions. Such administration routes include oral, rectal, sublingual, topical, nasal, transdermal or parenteral route. The expression “parenteral” includes subcutaneous, intravenous, epidural, irrigation, intramuscular, infusion or delivery pumps. Particularly, parenteral and nasal routes are preferred for the administration of the compositions claimed. in this invention.

For nasal administration, the active compounds may be dissolved in a pharmaceutical carrier and administered as a solution, emulsion, including micro- and. nanoemulsions, or suspension. Examples of suitable carriers are water and saline, or solid suspensions such as sprays, lactose, fructose or chitosan flakes. Other carriers may also contain other ingredients, for example preservatives, suspending agents, solubilizing agents, buffers and the like,

For parenteral administration, the active ingredients may also be dissolved in a pharmaceutical carrier and administered, as a solution, emulsion, including micro- and nanoemulsions, or suspension. Examples of suitable carriers are water, saline, dextrose solutions, fructose solutions or animal, vegetable or synthetic oils.

Properties of the Synergistic Immunogenic Compositions

The compositions of the present invention show an unexpected. synergistic effect on the immune response against the pneumococcal component. As can be seen in the Examples below, we have observed a significant increase in the level of antibodies induced against PspA and in the protection against lethal challenge with isolates from several pneumococcal strains. It was also observed that the combination of cell pertussis vaccine with a PspA antigen leads to a significant increase in the specific immune response against PspA, inducing a. more effective and unexpected protective response, which reinforces the hypothesis of a synergistic interaction between these two components.

Additionally, the synergistic immunogenic compositions of the present invention resulting from the combination of protein antigens of families 1 and 2 or fragments thereof exhibit the unexpected technical effect of causing a cross or heterologous immune response against S. pneumoniae strains which express surface proteins belonging to different clades and families from those present in the composition. In the experiments, a protein belonging to clade 5 and a protein belonging to clade 4, both from family 2 of pneumococcal PspA, were combined into a vaccine composition and this was inoculated in mice. After 21 days, the production of IgG antibodies which react with several pneumococcal strains expressing PspA of different clades of families 1 and 2 was observed.

Additionally, it was shown that the immunogenic compositions of the present invention promotes protection against different pneumococcal strains expressing different PspA in mice when used, via nasal route and that the addition of PV or PV_{L} significantly increases the production of IgA and IgG antibodies against these microorganisms.

Furthermore, the fact that the combination of PspA antigens and B. pertussis cell adjuvant can be further added to DIP vaccine, which is already administered in children in Brazil (at 2, 4 and 6 months old, with one booster at 15 months old and another between 4 and 6 years old), allows immunization against pneumococcal infections to the more susceptible population range.

Use of the Synergistic Immunogenic Compositions

Considering the properties of the synergistic immunogenic compositions of the present invention, the use of the synergistic immunogenic compositions for preventing infections caused by S. pneumoniae and B. pertussis in humans is a further aspect of the present invention.

The use of the combination of inactivated B. pertussis antigen adjuvant and PspA protein antigen with diphtheria and/or tetanus toxoids for preventing infections caused by S. pneumoniae, B. pertussis, tetanus and/or diphtheria in animals or humans is a further aspect of the present invention.

The use of one or trio re synergistic immunogenic compositions of the present invention in the manufacture of a combined vaccine for preventing whooping cough and infections caused by Streptococcus pneumoniae is a further aspect of the present invention.

The use of one or more synergistic immunogenic compositions of the present invention in the manufacture of a combined vaccine for preventing whooping cough, diphtheria and infections caused. by Streptococcus pneumoniae is a further aspect of the present invention.

The use of one or more synergistic immunogenic compositions of the present invention in the manufacture of a combined vaccine for preventing whooping cough, tetanus and infections caused by Streptococcus pneumoniae is a further aspect of the present invention.

The use of one or more synergistic immunogenic compositions of the present invention in the manufacture of a combined, vaccine for preventing whooping cough, tetanus, diphtheria and infections caused by Streptococcus pneumoniae is a further aspect of the present invention.

### EXAMPLES

In order to allow a better understanding of the present invention, and clearly demonstrate the technical improvements which were achieved, the results of the various tests carried out with regard to this invention are now given as examples.

Example 1 describes the obtaining of PspA antigen and the cell pertussis vaccines used are defined. Example 2 describes the properties and the use of vaccine combinations.

### Example 1

### Obtaining the PspA Antigen and Preparing the Immunogenic Compositions

a) Construction of vectors for expressing PspA. The encoding sequences from N-terminal region to proline-rich region of mature PspA of clade 1 (PspA1), clade 4 (PspA4Pro) and clade 5 (PspA5) were amplified, by polymerase chain reaction (PGR) from genomic DNA of S. pneumoniae isolates St 435/96, St 255/00 and St 122/02 (PspA clade 1, 4 and 5), respectively. Amplification was performed using the enzyme Taq DMA polymerase (Invitrogen) according- to the manufacturer's instructions and at the following temperature conditions: 94° C.-4 minutes/94° C.-1 minute/60° C.-1 minute/72° C.-2 minutes/72° C.-10 minutes/4° C., performing 30 amplification cycles. The following oligonucleotide pairs were used: pspA1 (SEQ. ID. No. 1), pspA4pro (SEQ. ID. No. 2) and pspA5 (SEQ. ID. No. 3).

The amplified fragments were separated by electrophoresis in 1% agarose gel and purified with the “GFX DNA and Gel Purification” kit. (GE-Healthcare). These purified fragments were used in the binding reaction into vector pGEM-T-Easy (Promega). The binding reactions were maintained at 4° C. for 16 hours and used for transforming Escherichia coli DH5α. The transformed bacteria were plated on 2YT-amp medium (Tryptone 1.6% (w/V) yeast extract 1% (w/V), NaCl 0.5% (w/V) and 100 μg/mL ampicillin) containing X-Gal (5-bromo-4-chloro-3-indol-β-D-galactopyranoside 0.008%) for the selection of white and blue colonies. The extraction of plasmid DNA from transformed colonies was performed with the GFX miniprep kit (GE-Healthcare). The presence of the insert was confirmed by restriction analysis and the sequence was determined by ABI Prism 3100 automated sequencer (PS Applied Biosystems). These digested samples were analyzed by electrophoresis in 1% agarose gel and the size bands corresponding to the expected containing the insert ut and purified. These inserts were bound to the expression vector in E. coli pAE, which allows the expression of recombinant protein fused with 6 histidine residues at the N-terminal portion, enabling its purification on nickel chelating column (Ramos C R. et al. Brazilian Journal of Medical and Biological Research 2004; 37:1103-1109). E. coli DH5α was transformed with the product of this bond and the plasmids were isolated by miniprep, producing pAE-pspA1, pAE-pspA4Pro and pAE-pspA5 vectors. The pspA1, pspA4 and pspA5 sequences were deposited at GenBank with the following accession numbers: AY082387, EF649969 and EF649970, respectively (Darrieux M. et al. Journal of Medical Microbiology 2008, 57:273-278). The original pspA4 fragment encodes a protein that comprises two proline repetition blocks separated by a proline free region in its C-terminal region, whereas pspA4Pro has just the first proline repetition block at this same region (Moreno A T, et al. Clinical find Vaccine Immunology 2010, 17:439-446).

b) Expression and purification of PspA. EL21 (SI) competent E. coli (Invitrogen) was transformed with pAE-pspA1, pAE-pspA4Pro and p&E-pspA5 constructed vectors and grown at 30° C. for 16 hours. The expression of 17 polymerase in this strain is under the control of the promoter inducible by pro-U osmotic shock, whereas expression of the cloned gene in pAE is under the control of the T7 promoter. Isolated clones were inoculated in 5 mL of 2YT/ON-amp medium (Tryptone 1.6% (w/V), yeast extract 1% (w/V) and 100 μg/mL ampicillin) and grown at 30° C. for 16 hours. These inoculates were diluted at a 1:30 ratio in the same medium with a final volume of 300 mL. The protein expression was induced by addition of NaCl (300 mM) and cultivation for 3 hours. The ceils were centrifuged at 3,200 g for 15 minutes, resuspended in 30 mL of balance buffer (50 mM Tris pH 8, 150 mM NaCl and 5 mM imidazole) and iysed in the French Press apparatus (1500 psi). The lysed cells were centrifuged at 12,900 g for 30 minutes at 4° C. and the supernatant was collected and filtered. Purification of the soluble fraction was carried out by nickel affinity chromatography with the aid of Akta Prime apparatus (GE Healthcare). After cell lysis, the supernatant was adsorbed into the column precharged with 300 mM NiSO₄ and balanced with 20 mL of balance buffer. Washes were performed with 50 mL of washing buffer (50 mM Tris pH 8, 150 mM NaCl and 50 mM Imidazole) and the proteins were recovered with the elution buffer (50 mM Tris pH 8, 150 mM NaCl and 250 mM Imidazole). After chromatography, the collected fractions were separated and analyzed by SDS-polyacrylamide gel 12% and then dialyzed using a dialysis buffer (10 mM Tris pH 8, 20 mM NaCl, 0.1% glycine). This solution was left under stirring for 16 hours at 4° C. The dosage of the recombinant proteins was determined by the Bradford method (Protein Assay Kit Biorad) using bovine serum albumin as standard. The proteins were stored at −20° C.

c) Preparation of insmunogenic compositions. For the preparation of immunogenic compositions and pertussis vaccine PV, a lyophilized B. pertussis 137 strain was resuspended in 0.9% saline, seeded, into Bordet-Gengou medium tubes and incubated at 35° C. tor 72 hours. The cell cultures were amplified by transference to other tubes containing Bordet-Gengou medium and incubated at 35° C. for 24 hours. The cell cultures were then inoculated into 500 mL Erlenmeyer flasks containing 100 mL of modified Stainer-Scholte medium and maintained, at 35° C. under stirring at 150 rpm. After 24 hours, the cultivation in liquid medium was inoculated in a 150 L pre-fermenter containing 60 L of culture medium. After 20 hours at 35° C., 40 L of culture were transferred to a 750 L fermenter containing 400 L of medium or 60 L of culture were transferred to a 1000 L fermenter containing 600 L of medium. After 20 hours, the culture was subjected to tangential filtration (0.22 μm) in order to obtain the bacterial biomass which was then detoxified with 0.2% formaldehyde. Detoxified B. pertussis cells were centrifuged at 3000 rpm for 1 hour at 4° C. or concentrated by tangential filtration. For the production of low pertussis vaccine (PV_{L}), the bacterial concentrate was maintained for 1 hour at room temperature under stirring in the presence of organic solvent (20 mL of 9% solvent to 1 g of wet bacterial mass). The suspension was centrifuged at 800 rpm for 1 hour at 4° C. or concentrated by tangential filtration. The final precipitate was rinsed with 0.9% saline (1 g precipitated in 10 mL of saline), followed by further centrifugation or tangential filtration. The final precipitate was resuspended in phosphate buffered saline (pH 6.8). These processes are described in the patent application related to the process of obtaining a novel cell pertussis vaccine (Patent Application no. PI 0402630-6). The immunogenic composition of the dual vaccine DT comprises diphtheria toxoid and tetanus toxoid. The immunogenic composition of DTP vaccine comprises the PV component. The immunogenic composition of the DTPL vaccine comprises the PVL pertussis component. The three vaccine immunogenic compositions use aluminum hydroxide as an adjuvant.

For the preparation of the tetanus toxoid component present in immunogenic compositions for DT, DTP and DTPL vaccines, the iyophilized strain of Harvard-Caracas Clostridium tetani was resuspended in Fluid. Thioglycollate medium. Two passages were made in the same medium which were incubated for 40 hours and 24 hours at 37° C. respectively. The culture from the second passage was transferred to a flask containing Fluid Thioglycollate medium, and incubated at 37° C. for 8 hours. The cultivation in the flask was used as a production inoculum for the fermenter. The culture medium IB for the production of Tetanus Toxin was prepared and sterilized by 0.22 μm membrane filtration. Subsequently, the Production Inoculum was aseptically added, to the fermenter. The fermentation process lasts 88 hours±1 hour at 36° C.±1° C. Then the cultivation was subjected to tangential filtration on 0.22 μm membranes for separating biomass and the toxic filtrate containing the Tetanus Toxin, which is collected in a 300 liter container. Tetanus Toxin was concentrated by molecular ultrafiltration system with a 30 kDa cutoff. Then a solution of 37% Formaldehyde p.a. in 1% ratio, Glycine (1 g per each 1.2 mL of formaldehyde) and 0.5% Sodium Bicarbonate was added. The concentrated Tetanus Toxin was

subjected to sterilizing filtration on 0.22 μm membranes. The sterile filtrated product was incubated at 36° C.±1° C. for 30 days. After this detoxification period, a sample was taken for biological testing (to verify the loss of toxicity) and for microbiological and physrcochemical testing. After release of the quality control tests, the product was purified by gel filtration chromatography (sephacril S-200). Then thimerosal was added as preservative and the product was subjected to sterilizing filtration. After approval of the quality control tests the product is used in the production of Dual Vaccine for children (DT), Dual Vaccine for adults (dT) and Triple Vaccine (DTP or DTP_{L}).

For the preparation of diphtheria toxoid component present in immunogenic compositions for DT, DTP and DTPL vaccines, the lyophilized strain of Park-Williams 8 Coryriecbacterium diphtherias was reconstituted in IB medium for producing diphtheria toxin and seeded in Loeffler medium. After 24 hours at 36° C.±1° C., the culture was transferred to 500 mL Erlenmeyer flasks containing 100 mL of IB medium for producing- diphtheria toxin, previously added with 40% calcium chloride solution. The Erlenmeyer flasks were kept for 24 hours at 36° C.±1° C. under sitrring at 150 rpm. Two further transferences were performed under the same conditions. The third culture was used as a Production Inoculum for the fermenter.

The culture medium IB for the production of diphtheria toxin was prepared, sterilized. by 0.22 μm membrane filtration and. added to a 4 0% calcium chloride solution. Subsequently, the Production Inoculum was aseptically added to the fermenter. The fermentation process lasts 64 hears±1 hour at 36° C.±1° C. Then the cultivation, was subjected to tangential filtration on 0.22 μm membranes for separating biomass and the toxic filtrate containing the Diphtheria Toxin, which is collected in a 300 liter container. Diphtheria Toxin was concentrated, by molecular ultrafiltration system with a 30 kDa cutoff. Then a solution of 37% Formaldehyde p.a. in a 0.7% ratio, 2 M L-Lysine solution and 0.5% Sodium Bicarbonate was added. The concentrated Diphtheria Toxin was subjected to sterilizing filtration on 0.22 μm membranes. The sterile filtrated product was incubated at 36° C.±1° C. for 30 days. After this detoxification period, a sample was taken for biological testing (to verify the loss of toxicity) and for microbiological and physicochemical testing. After release of the quality control tests, the product was purified by ammonium sulphate precipitation and subsequently diafiltered and concentrated in the tangential filtration system with a 30 kDa cutoff. Then thimerosal was added as preservative and the product was subjected to sterilizing filtration. After approval of the quality control tests the product was used in the production of Dual Vaccine for children (DT), Dual Vaccine for adults (dT) and Triple Vaccine (DTP or DTP_{L}).

### Example 2

### Properties of Immunogenic Compositions: Increase of Reactivity and Cross-Protection Against Pneumococcus by Using a Combination of PspA with Cell Pertusis Vaccines

a) Immunisation of Hiics and. lethal intranasal challenge. BALB/c mice were immunized with 5 μg of PspA4Pro or PspA5 alone or in combination with ⅛ of the PV or PVL human dose. Prior to formulating the compositions, residual LPS from E. coli was removed from protein preparations by extraction with Triton X-114 (Aida & Y. Pabst M J. Journal of Immunological Methods 1990, 132:191-195), Nasal administration was carried out in animals which were previously anesthetized intraperitoneally with 200 μL of a mixture of 0.2% xylazine hydrochloride (Syntec) and 0.5% ketamine hydrochloride (Syntec). Vaccines were administered in a 10 L volume on days 0, 3, 14, 17, 28 and 31 (total of 6 doses). In the subcutaneous immunization experiments, animals received one dose of a vaccine comprising 5 μg of PspA4Pro or PspA5 combined with ⅛ of the human dose of the dual vaccine DT or triple vaccine DTPL in a 100 mL volume. The mice were bled by retroorbital puncture and the serum was collected 21 days after the last immunization. Bronchoaiveoiar lavage fluid (EALF) samples were also collected. The animals were sacrificed by the injection of an urethane lethal dose (15 mg per 10 g body weight) and two washes were performed with 0.5 and 1 mL PBS by using a catheter inserted into the trachea. The collected fluid was aliquotea and kept at −80° C. The presence of anti-PspA in the serum and BALE was measured by an immunoassay (ELISA—“Enzymatic-linked immunoassay”). 96 well plates with flat bottom (Maxisorp-Nunc) were coated with recombinant protein solution (1 μg/mL) in Carbonate-Bicarbonate buffer (50 mmol/L Na₂CO₃ and 50 mmol/L Na2HPO4, pH 9.6) and maintained at 4° C. for 16 hours. Then the plates were maintained at 37° C. for 30 minutes and washed three times with PBS-T (1.37 mol/L NaCl, 27 mmol/L KCl, 100 mmol/L Na₂HPO₄, 14 mmol/L KH₂PO₄, pH 7.4 and 0.05% Tween 20). The blocks were made with PBS-FBS (PBS containing 10% fetal bovine serum) at 37° C. for 30 minutes. The plates were washed three times with PBS-T. Serial dilutions of serum from immunized mice or controls were added in PBS-FBS, followed by incubation at 37° C. for 1 hour and three more washes with PBS-T. Then, anti-mouse IgG antibody conjugated with peroxidase (Southern Biotech) in PBS-FBS was added to the plates followed by incubation at 37° C. for 1 hour. After this incubation, the plates were washed three times with PBS-T. The antibodies were detected by addition of substrate solution for development. Reactions were stopped with H2SO₄ to a 1.25 M final concentration and the absorbance at 492 nm (A_{492 nm}) was determined in an ELISA reader (Labsystems). A standard curve was generated using- mouse IgG (Southern Biotech) tor sensing the plates. The statistical analysis of the difference between the antibody concentrations was evaluated by Mann-Whitney U-test.

For the “immunoblotting” experiments, isolate 0603 (serotype 63, PspA from clade 1), isolate D39 (serotype 2, PspA from clade 2), isolate P2139 (serotype 6A, PspA from clade 2) , isolated TIGR4 (serotype 4, PspA from clade 3) and isolated ATCC6303 (serotype 3, PspA from clade 5) were used. Bacteria were grown on blood agar plates and subsequently in liquid THY medium (Todd-Hewitt medium containing 0.5% yeast extract—Difco) until DO_{600 nm} 0,6. The cells were centrifuged and the pellets were resuspended in lysis buffer—DOC (0.1% sodium deoxycholate, 0.01 sodium dodecyl sulfate (sds) and 0.15 M sodium citrate) and incubated at 37° C. for 10 minutes. Then the samples were centrifuged and the supernatant was collected and frozen at −20° C. Proteins were separated on 10% SDS-polyacrylamide gel electrophoresis (15 μg of protein were applied to the gel). Proteins were transferred to nitrocellulose membranes and incubated in PBS-T solution with 5% skimmed milk for 16 hours at 4° C. Membranes were incubated for 2 hours with serum containing anti-PspA antibodies (1/500) in PBS-T buffer containing 5% skframed milk. Then, the membranes were washed three times for 10 minutes with PBS-T, For immuno detection, 1 hour incubation was performed with diluted anti-mouse IgG antibody conjugated with peroxidase (Sigma) (1/1000) and the detection was performed with the ECL chemiluminescence kit (GE-Healthcare).

An invasive intranasal challenge was carried out in previously immunized mice. The animals were

intraperitoneally anesthetized with 200 μL of a mixture of 0.2% xylazine hydrochloride and 1.0% of ketamine hydrochloride. A66.1 (serotype 3, PspA from, clade 2) and ATCC6303 (serotype 3, PspA from clade 5) isolates were used. The isolates were cultured until the mid-log growth phase (DO_{600 nm}=0.4) in THY and aliquots were frozen at −80° C. The animals were then challenged by inoculation of 1×10⁶ CFU of the A66.1 isolate or 3×105 CFU of the ATCC6303 isolate in 50 μL of saline in one nostril with the aid of a micropipette. In passive protection experiments, the serum of immunized animals was inactivated at 56° C. for 30 minutes. Groups of 6 non-immunized BALB/c mice were inoculated with 500 μL of a 1:100 dilution of each serum via intraperitoneal route 2 hours before the challenge with ATCC6303 isolate. The survival of the animals was observed for 10 days and the difference in survival between experimental groups was assessed by Fisher's Exact Test. The amount of bacteria in the blood, and lungs of animals was further examined at different times after challenge. The lung tissue was broken in 1 mL of 0.45% saline (½ saline) by using a nylon mesh (“cell strainer”). Serial dilutions of blood, and lung homogenate were plated on blood agar and incubated for 18 hours at 37° C. for determination of CFU (colony forming unit). The detection of 0 CFU was considered as 1 CFU. The lower limit of detection was of 100 CFU/mL for blood samples and 5 CFU/animal for lung samples.

b) Protection induced by nasal immunization with an immunogenic composition comprising PspA and call pertussis vaccine. BALB/c mice were intranasally immunized with PspA5 only or with the combination with PV (PV-PspA5) and then subjected to a lethal intranasal challenge with the ATCC6303 isolate (PspA from clade 5). In this model, non-immunized animals were killed 72 hours after challenge (Ferreira D M. et al. Microbial Pathogenesis 2009, 47:157-163). In Table 1, we can observe that there was significant protection of animals in the group that received the PspA5-PV formulation, whereas there was only 50% survival in mice immunized with PspA5 only. No protection was observed in animals inoculated with PV only.

TABLE 1
Survival of BALB/c mice after intranasal challenge with
S. pneumoniae ATCC6303 - PV adjuvant property

| | alive/total | % survivors | P* |
|---|---|---|---|
| Unimmunized | 0/6 | — | |
| PV | 1/6 | 16.6 | 1 |
| PspA5 | 3/6 | 50 | 0.09 |
| PspA5-VP | 6/6 | 100 | 0.01 |

^{*}Fisher's Exact Test. The results represent three independent experiments.

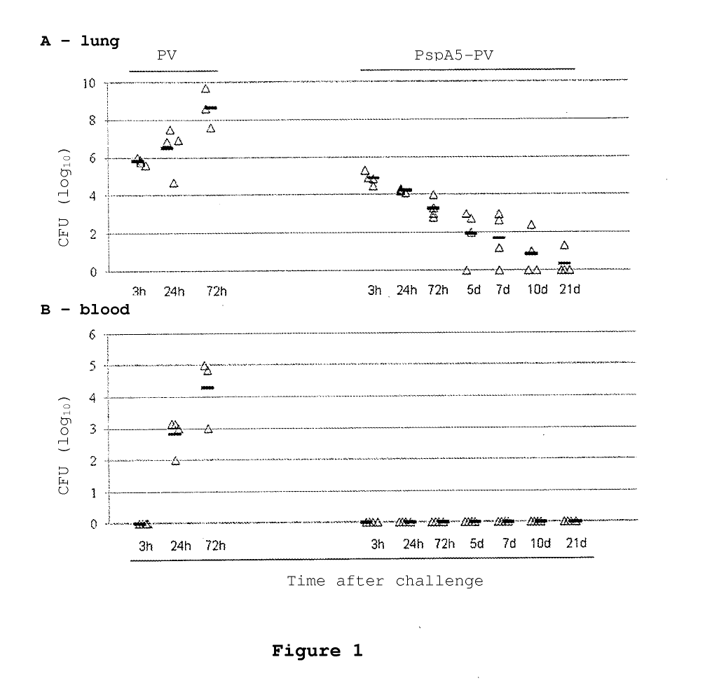

The presence of pneumococci in lung homogenates and blood serum of animals immunized with PV or PspA5-PV at different times after challenge was evaluated, As can be observed in FIG. 1A, large amounts of bacteria were recovered 72 hours after challenge in lungs of animals inoculated with PV. There was a decrease in the amount of pneumococcal recovered from the lungs of mice immunized with PspA5-PV in increasing times, resulting in the almost complete disappearance of bacteria 21 days after challenge. Moreover, it was not possible to detect pneumococci in the blood of animals inoculated with PspA5-PV, while mice immunized with PV only showed large amounts of bacteria in the blood 24 hours after challenge (FIG. 1B).

Since it has been demonstrated in clinical trial that the novel PV_{L} vaccine with lower content of LPS induces an immune response against pertussis which is similar to the conventional PV vaccine (Zorzeto T Q, et al. Clinical and Vaccine Immunology 2009, 16:544-550), immunization experiments were carried out by the combination of PspA5 with PV_{L} and intranasal challenge with the ATCC6303 isolate. As can be seen from Table 2, there was significant protection against infection by pneumococcus only in the group immunized with PspA5-PV_{L}. Again, the inoculation of PspA5 only induced partial protection with a 50% survival.

TABLE 2
Survival, of BALB/c mice after intranasal challenge with
S. pneumoniae ATCC6303 - PV_{L} adjuvant property.

| | alive/total | % survivors | P* |
|---|---|---|---|
| Unimmunized | 0/6 | — | — |
| VP_{L} | 0/5 | — | 1 |
| PspA5 | 3/6 | 50 | 0.09 |
| PspA5-VP_{L} | 5/6 | 83.3 | 0.007 |

*Fisher's Exact Test. The results represent two independent experiments.

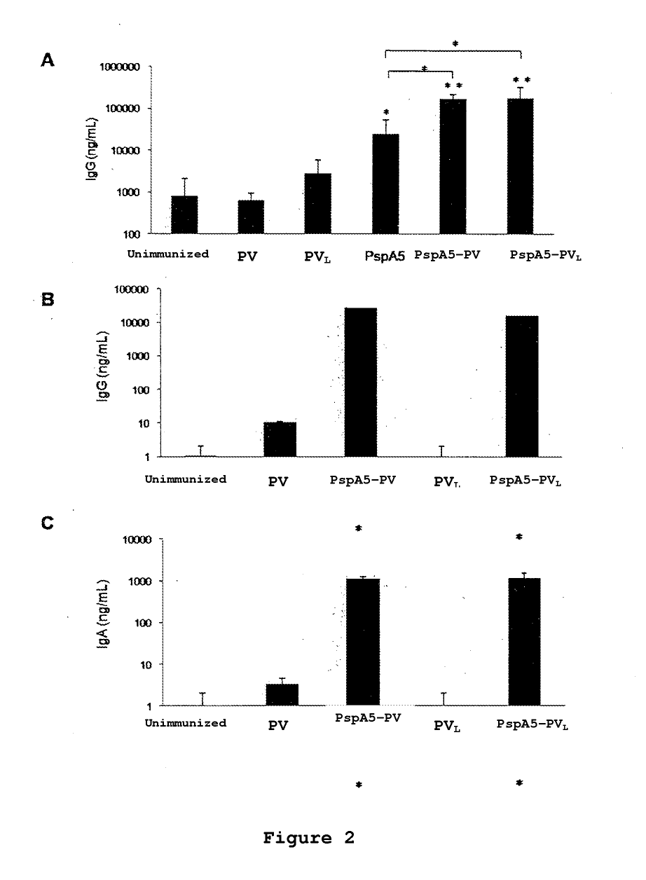

By evaluating the induction of specific antibodies, we found that nasal immunization with either PspA5-PV or PspA-PV_{L} formulation was able to induce very high levels of anti-PspA5 antibodies in the serum of mice. The concentration of specific antibodies was significantly higher in immunized groups than in control groups (P<0.05 in the comparison among control groups and PspAS, and P<0.005 in the comparison among control groups and PspAS-PV or PspA5-PV_{L}). Significant differences were also observed when comparing the serum of animals immunized with PspAS-PV or PspA5-PV_{L} and the PspAS group (P<0.05) (FIG. 2A). Moreover, both PspA5-PV and PspAS-PV_{L} formulations were able to induce high levels of anti-PspA IgG and IgA in BALF samples (FIG. 2B and 2C).

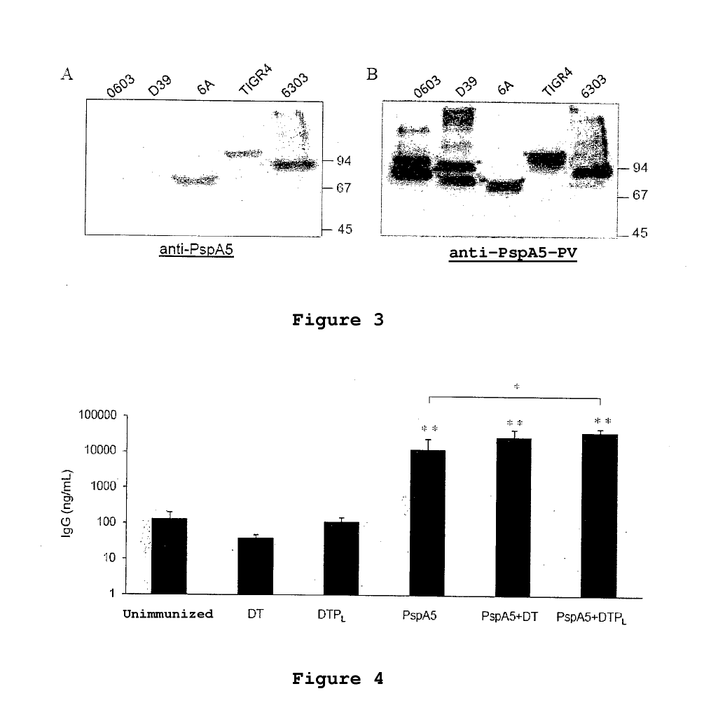

It was demonstrated that serum produced against PspA5 shows cross reactivity with PspA from different clades and families (Darrieux M. et al. Journal of Medical Microbiology 2008; 57:273-278, Moreno A T, et al. Clinical and Vaccine Immunology 2010; 17: 439-446). When evaluating a panel of isolates commonly used in mice challenge models with pneumococci, it was possible to confirm that the serum, of animals intranasally immunized with PspA5 is able to recognize total extract of different isolates in “immunoblotting” experiment (FIG. 3A). However, the serum of mice immunized with the PspA5-PV formulation presents an even higher cross-reactivity as a result of high levels of anti-PspA5 antibodies induced by the combined vaccine (FIG. 3B).

c) Cross protection induced by parenteral inoculation of immunogenic compositions comprising PspA and DTP_{L}. As the previous experiments indicated that the combination of PspA with a cell pertussis vaccine is able to induce protection against infections caused by pneumococci, the possibility of using the combination of PspA5 with the DTP_{L} vaccine by subcutaneous route in mice was tested. After a single immunization with 5 μg of PspA alone or in combination with DT or DTP_{L}, high levels of antibodies were detected (FIG. 4) (P=0.002 in the comparison among PspA5, PspA5-DT or PspA5-DTP_{L} and non-immunized animals, and P=0.002 in the comparison among PspAS-DT or PspAS-DTP_{L} and DT ou DTP_{L}, respectively). The PspA5-DTP_{L} formulation has also induced higher levels of antibodies if compared to the group immunized with PspA5 only (P=0.008). Moreover, 100% survival of animals immunized with a dose of PspA5-DTPL after challenge with ATCC6303 (P=0.001 compared with control groups) was observed. Significant protection was also observed with the formulation PspA5-DT, with 66% survival (P=0.03, compared with control group), as shown in Table 3.

TABLE 3
Survival of BALB/c mice alter invasive intranasal
challenge with S. pneumoniae A66.1 and ATCC6303
after subcutaneous immunization with PspA5

| | ATCC6303 | | | A66.1 | | |
|---|---|---|---|---|---|---|
| | (serotype 3, PspA5) | | | (serotype 3, PspA2) | | |
| | alive/ | % | | alive/ | % | |
| | total | survivors | P* | total | survivors | P* |
| Unimmunized | 0/6 | — | — | 0/5 | — | — |
| DT | 0/6 | — | — | 0/6 | — | — |
| DTP_{L} | 0/6 | — | — | 0/6 | — | — |
| PspA5 | 2/6 | 33.3 | 0.22 | 0/6 | — | — |
| PspA5 + DT | 4/6 | 66.6 | 0.03 | 3/6 | 50 | 0.09 |
| PspA5 + DTP_{L} | 6/6 | 100 | 0.001 | 4/6 | 66.6 | 0.03 |

*Fisher's Exact Test

When performing a challenge with the A66.1 isolate, significant protection was observed only in the group immunized with. the PspA5-DTP_{L} formulation (P=0.03, in comparison with the control group) (Table 3). Therefore, heterologous protection was observed since PspA5 belongs to family 2 and A66.1 bacterium expresses PspA of clade 2, belonging to family 1. It is important to emphasize that the survival of 66% of the animals after challenge with this isolate was obtained after a single subcutaneous immunization with PspA5-DTP_{L} and that higher protection levels can be achieved by administering booster doses.

PspA4Pro is the other PspA fragment that had shown ability to induce antibodies with high cross-reactivity with different pneumococcal isolates (Darrieux M. et al. Journal of Medical Microbiology 2008; 57:273-278, Moreno A T. et al. Clinical and Vaccine Immunology 2010, 17:439-446). Thus, immunization with a subcutaneous dose of PspA4Pro alone or in combination with DTPL was also tested. In Table 4, it can be observed that only the groups immunized with PspA4Pro-DTP_{L} have shown significant protection (P=0.05, in comparison with the control group).

TABLE 4
Survival of BALB/c mice after invasive intranasal
challenge with S. pneumoniae A66.1 and ATCC6303
after subcutaneous immunization with PspA4Pro

| | ATCC6303 | | | A66.1 | | |
|---|---|---|---|---|---|---|
| | (serotype 3, PspA5) | | | (serotype 3, PspA2) | | |
| | alive/ | % | | alive/ | % | |
| | total | survivors | P* | total | survivors | P* |
| Unimmunized | 1/6 | — | — | 0/5 | — | — |
| DTP_{L} | 0/6 | — | — | 0/6 | — | — |
| PspA4Pro | 0/6 | — | — | 0/6 | — | — |
| PspA4Pro + DTP_{L} | 5/6 | 83.3 | 0.05 | 4/6 | 66.6 | 0.05 |

*Fisher's Exact Test

As PspA4Pro belongs to family 2, homologous protection was observed again with a bacterium that expresses PspA of family 2 (ATCC6303) and heterologous protection was observed, with a bacterium that expresses PspA of family 1 (A56.1). As it was observed for PspA5-DTP_{L}, the survival of 66% of animals was obtained after a single immunization with PspA4Pro-DTP_{L} and higher protection levels are expected after administration of booster doses.

1. Synergistic immunogenic composition characterized by comprising at least one cell antigen of inactivated Bordetella pertussis and at least one protein antigen comprising one or more surface protein A of Streptococcus pneumoniae or fragments thereof, and a pharmaceutically acceptable carrier.

2. Synergistic immunogenic composition according to claim 1, characterized in that the cell antigen of Bordetella pertussis is inactivated Bordetella pertussis bacteria comprising low contents of lipopolysaccharides.

3. Synergistic immunogenic composition according to claim 1, characterized in that the Surface Proteins A of Streptococcus pneumoniae or fragments thereof comprise a mixture of at least two clades of Surface Protein A.

4. Synergistic immunogenic composition according to claim 1, characterized in that the Surface Protein A of Streptococcus pneumoniae or fragments thereof are selected from clades belonging to families 1 and 2.

5. Synergistic immunogenic composition according to claim 4, characterized in that the Surface Proteins A of Streptococcus pneumoniae or fragments thereof are selected from clades 1, 4 and 5.

6. Synergistic immunogenic composition according to claim 5, characterized in that the Surface Proteins A of Streptococcus pneumoniae or fragments thereof belong to clade 1.

7. Synergistic immunogenic composition according to claim 4, characterized in that the Surface Proteins A of Streptococcus pneumoniae or fragments thereof belong to clade 4.

8. Synergistic immunogenic composition according to claim 5, characterized in that the Surface Proteins A of Streptococcus pneumoniae or fragments thereof belong to clade 5.

9. Synergistic immunogenic composition according to claim 1, characterized in that the Surface Proteins A fragments of Streptococcus pneumoniae are selected from the group consisting of: α-helix N-terminal region and region rich in proline residues.

10. Synergistic immunogenic composition according to claim 1, characterized in that the Surface Protein A fragment of Streptococcus pneumoniae comprises the N-terminal region.

11. Synergistic immunogenic composition according to claim 1, characterized in that the protein antigen comprises two or more Surface Protein A fragments of Streptococcus pneumoniae which may be fused to form a hybrid protein.

12. Synergistic immunogenic composition according to claim 11, characterized in that the fused Surface Protein A fragments of Streptococcus pneumoniae belong to clades 1, 4 or 5.

13. Synergistic immunogenic composition according to claim 1, characterized in that the mixture of Surface Protein A of Streptococcus pneumoniae or fragments thereof provides heterologous protection against antigens from other clades of Surface Protein A of Streptococcus pneumoniae which are not present in the mixture.

14. Synergistic immunogenic composition according to claim 1, characterized by further comprising one or more antigens of inactivated toxins.

15. Synergistic immunogenic composition according to claim 14, characterized by the fact that the antigen is selected from the group consisting of inactivated diphtheria toxin and inactivated tetanus toxin.

16. Synergistic immunogenic composition according to claim 14, characterized by the fact that the antigen is inactivated diphtheria toxin.

17. Synergistic immunogenic composition according to claim 14, characterized by the fact that the antigen is inactivated tetanus toxin.

18. Synergistic immunogenic composition according to claim 1, characterized by the fact that it is for nasal administration.

19. Synergistic immunogenic composition according to claim 1, characterized by the fact that it is for parenteral administration.

20-22. (canceled)


SEQUENCE LISTING (3 sequences)

SEQ ID NO: 1 - DNA, 56 bp, Streptococcus pneumoniae
tagctcgaggaagaagcgcccgtagctagttatctagattttggtgcaggagctgg

SEQ ID NO: 2 - DNA, 61 bp, Streptococcus pneumoniae
tagctcgagaccatggtaagagcagaagaagccggtaccttatggttttggtgctggagct

SEQ ID NO: 3 - DNA, 63 bp, Streptococcus pneumoniae
tagctcgagaccatggtaagagcagaagaagcctagttatctagattttggtgcaggagctgg